(19) United States
(12) Patent Application Publication
(10) Pub. No.: US 2007/0184122 A1
(43) Pub. Date: Aug. 9, 2007

(54) FIBRONECTIN-MODIFIED ECM TISSUE GRAFT CONSTRUCTS AND METHODS FOR PREPARATION AND USE THEREOF

(76) Inventors: Chad E. Johnson, West Lafayette, IN (US); Edith S. Nihsen, Lafayette, IN (US); David A. Zopf, Plymouth, MI (US); Abram D. Janis, Stevenson Ranch, CA (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/735,215

(22) Filed: Apr. 13, 2007

Related U.S. Application Data
(63) Continuation of application No. PCT/US05/36773, filed on Oct. 14, 2005.
(60) Provisional application No. 60/618,965, filed on Oct. 15, 2004.

(51) Int. Cl. A61K 35/37; A61K 31/727; A61K 38/18

(52) U.S. Cl. 424/550; 514/12; 514/56

(57) ABSTRACT

Described are modified submucosa and other extracellular matrix materials incorporating an amount of bound, exogenous fibronectin. Further described are such materials also having an amount of exogenous heparin bound to the exogenous fibronectin, and also potentially an amount of an exogenous bioactive material, such as a growth factor, bound to the exogenous heparin. Such materials may be used in methods for the treatment of wounds in patients.

### BACKGROUND

The present invention relates generally to tissue graft materials, and in particular aspects to tissue graft constructs including a submucosa or other extracellular matrix materials having exogenous fibronectin molecules bound thereto, and potentially also incorporating exogenous heparin and one or more exogenous growth factors. Such materials are useful in wound care and especially in the treatment of chronic wounds such as chronic ulcers.

As further background, wound healing is a complex process involving platelets, the immune system, the extracellular matrix, and various cytokines and growth factors. Dermal wound healing is especially critical to maintaining the body's primary line of defense. The skin provides the body with a protective barrier from chemical and mechanical challenges, harmful pathogens, and ultraviolet radiation. Chronic wounds compromise the skin's ability to defend against these agents, due to the prolonged wound healing process.

For chronic wounds, the body is unable to complete the wound healing process due to compromised vascularization or immune system. Without clinical intervention, these chronic wounds can lead to the spread of infection, significant necrotic tissue, and possible amputation in the case of ulcers in the foot. Advanced states of chronic dermal wounds present a significant clinical challenge. In the United States alone, there are over 3 million cases of chronic wounds annually.

In view of this background, there remain needs for improved or alternative medical grafting materials, methods for manufacturing medical grafting materials, and methods for using medical grafting materials. The present invention is addressed to these needs.

### DESCRIPTION OF THE FIGURES

FIG. 1: SIS released heparin, albumin, or fibronectin into buffered rinse solutions in a molecule specific manner. Heparin absorbed into SIS was removed by rinses with an ionic buffer, suggesting weak association with the matrix. Albumin absorbed into SIS and was largely retained through rinses, suggesting non-specific binding. In contrast, fibronectin absorbed into SIS and was significantly washed out in the first rinse with greatly diminished levels in subsequent rinses. This result suggests that loosely associated fibronectin was removed in the first rinse, but that the remaining fibronectin was firmly absorbed, likely due to specific binding. Columns represent the mean amount of each molecule normalized by the initial incubation solution. Error bars indicate SEM.

FIG. 2: Fibronectin content before and after incubation in a fibronectin solution. SIS has the ability to absorb and retain human plasma fibronectin from solution. Error bars equal one standard deviation. *p<0.05 vs. initial SIS. †p.05 vs. post-incubation SIS

### SUMMARY OF THE INVENTION

In one aspect, the present invention provides a medical graft material that includes submucosa or other remodelable extracellular matrix material, and exogenous fibronectin bound to the submucosa or other remodelable extracellular matrix material.

In another aspect, the present invention provides a method for preparing a medical graft material. The method includes contacting submucosa with a liquid medium containing fibronectin so as to prepare a modified submucosa material incorporating fibronectin specifically bound to the submucosa and fibronectin that is not specifically bound to the submucosa. The modified submucosa is rinsed so as to remove at least a portion of the fibronectin that is not specifically bound to the submucosa.

In another aspect, the invention provides a medical graft material that includes a collagenous extracellular matrix material having exogenous fibronectin molecules bound to the collagenous extracellular matrix material. Exogenous heparin and/or heparin sulfate molecules (sometimes together referred to herein as heparin(sulfate)) are bound to the exogenous fibronectin molecules, and exogenous bioactive molecules are bound to the exogenous heparin(sulfate) molecules. The exogenous bioactive molecules can be heparin(sulfate)-binding proteins such as heparin(sulfate)-binding growth factors.

In another embodiment, the present invention provides a method for treating a wound that includes contacting the wound with an extracellular matrix material having exogenous fibronectin molecules bound thereto.

The invention also provides a method for preparing a modified extracellular matrix material. The method includes the steps of: (a) providing an extracellular matrix material; (b) contacting the extracellular matrix material with an amount of exogenous fibronectin so as to prepare a first modified extracellular matrix material having fibronectin molecules bound to the extracellular matrix material; and (c) contacting the first modified extracellular matrix material with an amount of exogenous heparin and/or heparin sulfate so as to prepare a second modified extracellular matrix material having exogenous heparin(sulfate) molecules bound to the exogenous fibronectin. In certain forms, this method also includes the step of contacting the second modified extracellular matrix material with an amount of a bioactive substance that binds to heparin(sulfate), so as to prepare a third modified extracellular matrix material having molecules of the bioactive substance bound to the exogenous heparin(sulfate). The bioactive substance can be a heparin(sulfate)-binding protein, such as a heparin(sulfate)-binding growth factor. Many such bioactive substances that bind with affinity to heparin(sulfate) are known and can be used.

Additional embodiments, as well as features and advantages of the invention will be apparent to those of ordinary skill in the art from the descriptions herein.

### DETAILED DESCRIPTION

For the purposes of promoting an understanding of the principles of the invention, reference will now be made to certain embodiments and specific language will be used to describe the same. It will nevertheless be understood that no limitation of the scope of the invention is thereby intended, such alterations and further modifications in the described embodiments, and such further applications of the principles of the invention as illustrated therein being contemplated as would normally occur to one skilled in the art to which the invention relates.

As disclosed above, in certain aspects, the present invention provides fibronectin-modified extracellular matrix medical graft materials, especially fibronectin-modified submucosa medical graft materials, as well as methods for preparation and use of these materials. Such fibronectin-modified materials can be further modified with other bioactive molecules such as heparin and/or heparin sulfate, and in certain embodiments also proteins or other bioactive materials that bind to the heparin and/or heparin sulfate, especially growth factors.

In certain aspects of the invention, tissue graft materials are provided that incorporate an extracellular matrix material (ECM) and especially a submucosa material. Other ECM materials that may be used include renal capsule membrane, dura mater, pericardium, serosa, peritoneum, or basement membrane. Preferred medical graft products of the invention will include submucosa, such as submucosa derived from a warm-blooded vertebrate. Mammalian submucosa materials are preferred. In particular, submucosa materials derived from animals raised for meat or other product production, e.g. pigs, cattle or sheep, will be advantageous. Porcine submucosa provides a particularly preferred material for use in the present invention, especially porcine small intestine submucosa, more especially porcine small intestine submucosa retaining substantially its native cross-linking.

The submucosa or other ECM material can be derived from any suitable organ or other biological structure, including for example submucosa derived from the alimentary, respiratory, intestinal, urinary or genital tracts of warm-blooded vertebrates. Submucosa useful in the present invention can be obtained by harvesting such tissue sources and delaminating the submucosa from smooth muscle layers, mucosal layers, and/or other layers occurring in the tissue source. For additional information as to submucosal and other ECM materials useful in the present invention, and their isolation and treatment, reference can be made, for example, to U.S. Pat. Nos. 4,902,508, 5,554,389, 5,993,844, 6,206,931, and 6,099,567, each of which is incorporated herein by reference.

As prepared, the submucosa or other ECM material desirably retains growth factors or other bioactive components native to the source tissue. For example, the matrix material may include one or more growth factors such as basic fibroblast growth factor (FGF-2), transforming growth factor beta (TGF-beta), epidermal growth factor (EGF), and/or platelet derived growth factor (PDGF). As well, submucosa or other ECM material of the invention may include other biological materials such as heparin, heparin sulfate, hyaluronic acid, fibronectin and the like. Thus, generally spreading, the ECM material may retain one or more bioactive components from the tissue source that induces, directly or indirectly, a cellular response such as a change in cell morphology, proliferation, growth, protein or gene expression.

Alternatively, as prepared, the submucosa or other ECM material may be processed sufficiently with solutions of detergents, acids, bases, salts and/or other agents to remove essentially all growth factors or other bioactive components native to the source tissue, e.g. leaving an ECM substrate consisting essentially of collagen or of collagen and elastin (with elastin, when present, usually making up a minor amount of the material). For instance, in certain embodiments, the processed ECM substrate will be constituted at least about 95% by weight (dry) of collagen or a collagen/elastin combination, for example from about 98% to about 100% by dry weight of collagen or a collagen/elastin combination. Such an ECM substrate can then be modified with fibronectin and potentially other bioactive molecules as described herein. Still further, a suitable collageneous matrix material, to be modified with fibronectin and potentially the other bioactive molecules as discussed herein, can be prepared by reconstituting collagen, electroprocessing collagen (including e.g. electrospinning), or otherwise re-assembling collagen materials to form a collagenous matrix scaffold starting material. These and other aspects of preparing a suitable biocompatible substrate capable of binding fibronectin for use herein will be apparent to those skilled in the art.

ECM material used in the invention is preferably highly purified, for example, as described in U.S. Pat. No. 6,206,931. Thus, preferred material will exhibit an endotoxin level of less than about 12 endotoxin units (EU) per gram, more preferably less than about 5 EU per gram, and most preferably less than about 1 EU per gram. As additional preferences, the ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plate forming units (PFU) per grain, more preferably less than about 5 PFU per gram. These and additional properties of submucosa taught in U.S. Pat. No. 6,206,931 may be characteristic of the ECM material used in the present invention.

ECM materials used in the invention may be free of additional, non-native crosslinking, or may contain additional crosslinking. Such additional crosslinking may be achieved by photo-crosslinking techniques, by chemical crosslinkers, or by protein crosslinking induced by dehydration or other means. Chemical crosslinkers that may be used include for example aldehydes such as glutaraldehydes, diimides such as carbodiimides, e.g., 1-ethyl-3-(3-dimethylaminopropyl)carbodiimide hydrochloride, ribose or other sugars, acyl-azide, sulfo-N-hydroxysuccinamide, or polyepoxide compounds including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from 2 to about 10 epoxide groups per molecule.

It is also possible for an ECM material used in the invention to comprise a multilaminate ECM material. To form a multilaminate material, two or more ECM segments are stacked, or one ECM segment is folded over itself at least one time, and then the layers are fused or bonded together using a bonding technique, such as chemical cross-linking or vacuum pressing during dehydrating conditions.

An adhesive, glue or other bonding agent may also be used in achieving a bond between ECM layers. Suitable bonding agents may include, for example, collagen gels or pastes, gelatin, or other agents including reactive monomers or polymers, for example cyanoacrylate adhesives. As well, bonding can be achieved or facilitated using chemical cross-linking agents, such as glutaraldehyde, formaldehyde, epoxides, genipin or derivatives thereof, carbodiimide compounds, polyepoxide compounds, or other similar agents, including those others identified in the discussions above. Cross-linking of ECM materials can also be catalyzed by exposing the matrix to UV radiation, by treating the collagen-based matrix with enzymes such as transglutaminase and lysyl oxidase, and by photocross-linking. The combination of one or more of these with dehydration-induced bonding may also be used.

A variety of dehydration-induced bonding methods can be used to fuse ECM portions of the bioremodelable material. In one preferred embodiment, the multiple layers of ECM material are compressed under dehydrating conditions. The term “dehydrating conditions” is defined to include any mechanical or environmental condition which promotes or induces the removal of water from the ECM material. To promote dehydration of the compressed ECM material, at least one of the two surfaces compressing the matrix structure can be water permeable. Dehydration of the ECM material can optionally be further enhanced by applying blotting material, heating the matrix structure or blowing air, or other inert gas, across the exterior of the compressing surfaces. One particularly useful method of dehydration bonding ECM materials is lyophilization, e.g. freeze-drying or evaporative cooling conditions.

Another method of dehydration bonding comprises pulling a vacuum on the assembly while simultaneously pressing the assembly together. This method is known as vacuum pressing. During vacuum pressing, dehydration of the ECM materials in forced contact with one another effectively bonds the materials to one another, even in the absence of other agents for achieving a bond, although such agents can be used while also taking advantage at least in part of the dehydration-induced bonding. With sufficient compression and dehydration, the ECM materials can be caused to form a generally unitary ECM structure.

It is advantageous in some aspects of the invention to perform drying operations under relatively mild temperature exposure conditions that minimize deleterious effects upon the ECM materials of the invention, for example native collagen structures and potentially bioactive substances present. Thus, drying operations conducted with no or substantially no duration of exposure to temperatures above human body temperature or slightly higher, say, no higher than about 38° C., will preferably be used in some forms of the present invention. These include, for example, vacuum pressing operations at less than about 38° C., forced air drying at less than about 38° C., or either of these processes with no active heating—at about room temperature (about 25° C.) or with cooling. Relatively low temperature conditions also, of course, include lyophilization conditions.

Exogenous fibronectin materials (i.e. those derived separately from the ECM material being treated) useful in the invention include proteins comprising a native fibronectin polypeptide chain, whether isolated from naturally-occurring sources, or produced by recombinant DNA or other synthetic techniques, and includes allelic and phylogenetic counterpart variants of these proteins, as well as muteins thereof including truncated forms and deletion or addition mutants. Such muteins when used in the invention will desirably retain the ability to bind heparin or heparin sulfate and/or retain some level of chemotactic or cell adhesion activity exhibited by the native fibronectin protein.

To incorporate the fibronectin material into the ECM material, the ECM material will be suitably contacted with the fibronectin material. This is preferably achieved by contacting the ECM material with an aqueous solution of the fibronectin material for a period of time sufficient to bind a substantial amount of fibronectin to the ECM material. This contact time may vary, for example, from a few seconds to several hours, depending upon the circumstances.

After contact with the fibronectin solution or other source, the ECM material can optionally be rinsed with an aqueous medium or other suitable rinse liquid to remove essentially all or a portion of the non-bound or loosely-bound fibronectin. This rinse process may be conducted in a variety of ways to remove the non- or loosely-bound fibronectin to the desired extent. In certain embodiments of the invention, the rinse will be conducted sufficiently such that a predominant amount (greater than 50%) of the fibronectin molecules remaining incorporated in or on the matrix are stably or specifically bound, for example wherein greater than 50% of the fibronectin molecules are retained in the ECM material upon rinsing for one hour in phosphate buffered saline. In still other embodiments, at least about 75%, at least about 90% or essentially all (e.g. about 98% to 100%) of the fibronectin molecules remaining in or on the extracellular matrix material can be stably or specifically bound.

A fibronectin-modified submucosa or other ECM material as discussed above may be used itself as a tissue graft material in wound healing or other applications. In other embodiments of the invention, the fibronectin-modified ECM material is treated with at least one additional exogenous bioactive material. For example, inventive embodiments are provided wherein the fibronectin-modified ECM material is treated with exogenous heparin and/or exogenous heparin sulfate under conditions wherein the heparin or heparin sulfate binds to amounts of the exogenous fibronectin which in turn are bound to the ECM substrate. In some embodiments, sufficient amounts of heparin or heparin sulfate are thus incorporated with the ECM material so as to decrease the thrombogenicity of the ECM material. Such ECM materials may, for example, be used in vascular grafting applications where contact with blood of the patient will be encountered.

In still further embodiments of the invention, an ECM material modified with both fibronectin and heparin or heparin sulfate as discussed above will be modified with a third exogenous bioactive material, especially one that has the capacity to bind to the exogenous heparin or heparin sulfate. In this fashion, an ECM material having bound-(fibronectin)-(heparin and/or heparin sulfate)-(third exogenous bioactive molecule) ligands or moieties can be prepared. In this regard, a variety of suitable bioactive molecules that bind to heparin or heparin sulfate are known. These include, for example, Fibroblast Growth Factors (FGFs) such as FGF-1 (aFGF), FGF-2 (bFGF), FTF-3, FGF-4, FGF-5, FGF-6, FGF-7, PGF-8, and FGF-9; heparin binding epidermal growth factor (HBEFG); vascular endothelial growth factor (VEGF); placental growth factor (PIGF); heparin-binding EGF-like growth factor; transforming growth factor-beta (TFG-beta); interferon-gamma (IFN-gamma); platelet-derived growth factor (PDGF); pleiotrophin; platelet factor-4 (PF-4); interleukin-8 (IL-8); macrophage inflammatory protein-1 (MIP-1); interferon-γ-inducible protein-10 (IP-10); adhesive matrix proteins such as fibronectin, vitronectin, laminin, collagens, and thrombospondin; serine protease inhibitors such as antithrombin III, heparin co-factor II and protease nexins; and tumor necrosis factor. Other bioactive molecules that bind heparin and/or heparin sulfate are also known and can be used within the scope of the present invention. One of these bioactive materials, or a plurality (two or more) of these biomaterials, may be incorporated into a modified ECM graft material as disclosed herein. Heparin-binding growth factors, particularly those that promote or facilitate wound healing, provide a preferred set of exogenous bioactive materials for binding to exogenous heparin or heparin sulfate incorporated in or on the ECM material as discussed above. Other bioactive materials that bind with affinity to heparin or heparin sulfate may also be used for these purposes.

In certain embodiments of the invention, a fibronectin-modified ECM material will be rinsed to remove amounts of (including essentially all or a portion of) non- or loosely-bound fibronectin prior to treatment with the heparin and/or heparin sulfate, and the resulting heparin and/or heparin-sulfate modified ECM material will in turn be rinsed to remove amounts of (including essentially all or a portion of) non- or loosely-bound heparin and/or heparin sulfate. Thereafter, the ECM material will be treated with a third, heparin-binding exogenous bioactive material, e.g. one as discussed hereinabove, to prepare an ECM material having the -(fibronectin)-(heparin or heparin sulfate)-(bioactive molecule) ligands. The resulting material can then optionally also be rinsed to remove amounts of (including essentially all or a portion of) non- or loosely-bound heparin(sulfate)-binding exogenous bioactive material, to result in a material having substantial bound amounts of the heparin(sulfate)-binding exogenous bioactive material, e.g. with more bound than unbound material. Such manufacturing processes and the resulting material can be used to deliver the exogenous heparin(sulfate)-binding bioactive material to the graft site retained in close association with the ECM substrate material.

The exogenous fibronectin, heparin and/or heparin sulfate, and/or any further bioactive material used to modify the ECM material may each be from the same species of animal from which the ECM material was derived (e.g. autologous or allogenic relative to the ECM material) or may each be from a different species from the ECM material source (xenogenic relative to the ECM material). In certain embodiments, the ECM material will be xenogenic or allogenic relative to the patient receiving the graft, and the added exogenous material(s) will be from the same species (e.g. autologous or allogenic) as the patient receiving the graft. Illustratively, human patients may be treated with xenogenic or allogenic ECM materials (e.g. porcine-, bovine- or ovine-derived) that have been modified with exogenous human material(s) as described herein, those exogenous materials being naturally derived and/or recombinantly produced.

The relative amounts of the exogenous bioactive material(s) applied to the ECM material can be varied to modulate the properties of the resulting graft material. For example, in constructs incorporating fibronectin in combination with heparin and/or heparin sulfate, sufficient of the latter material(s) can be added to bind to and occupy all of the fibronectin, or lesser amounts can be added so as to provide a material having some unoccupied, exposed fibronectin molecules to facilitate providing or enhancing a chemotactic or cell attracting activity of the material. Similarly, the amounts of heparin or heparin-sulfate binding bioactive material (e.g. growth factor) can be varied to leave some heparin(sulfate) molecules unoccupied, or to bind to and consume essentially all heparin molecules available. In certain embodiments, ECM materials will be modified with exogenous fibronectin, an amount of exogenous heparin and/or heparin sulfate insufficient to occupy all of the fibronectin, and an amount of a heparin or heparin sulfate-binding bioactive material(s) (e.g. growth factor(s)) that is insufficient to occupy all of the heparin and/or heparin sulfate available. In this manner, beneficial amounts of all three (or more) added, exogenous materials can be immediately presented to a graft site receiving the graft material. In one non-limiting, illustrative example, the ratio of (fibronectin):(heparin and/or heparin sulfate):(growth factor or other heparin-binding substance) molecules added can be about 4:2:1.

Tissue graft materials of the present invention may be provided in a variety of forms, including for example in sheet form, particulate form, or fluidized (e.g. injectable) form. Sheet forms may include openings such as perforations, holes or slits, which may provide benefit in a variety of tissue grafting applications including in wound care grafting applications. The tissue graft materials can be in their final physical form or in a precursor form during treatment with exogenous fibronectin, exogenous heparin or heparin sulfate, and/or other exogenous bioactive molecules as disclosed herein. For example, a sheet of ECM material can be treated to have bound fibronectin, bound-(fibronectin)-(heparin or heparin sulfate) ligands, or bound-(fibronectin)-(heparin or heparin sulfate)-(third exogenous bioactive molecule) ligands, and thereafter modified in form. For example, subsequent modifications can include forming openings in the material as discussed above, or reducing the sheet material to particulate or fluidized (e.g. injectable) form. On the other hand, the a sheet of ECM material can first be modified with openings or to provide a particulate or fluidized form, and then modified to incorporate the bound fibronectin, bound-(fibronectin)-(heparin or heparin sulfate) ligands, or bound-(fibronectin)-(heparin or heparin sulfate)-(third exogenous bioactive molecule) ligands. Still further, one or more of these exogenous materials may be added to the ECM material in sheet form, and others added after modification of the sheet form e.g. to create openings, a particulate form, or a fluidized form. As those skilled in the art will appreciate, these and other modification techniques will be suitable for the present invention.

In certain inventive embodiments, a modified ECM in accordance with the invention is provided in a meshed form. Thus, the ECM medical graft product will have multiple slits therein to provide the mesh pattern, and in turn the mesh pattern will provide deformability to the collagen-containing layer, for example exhibiting an expansion ratio of at least about 1.2:1 when hydrated. These constructs will provide particular advantage in the treatment of externally exposed wounds such as burn wounds or ulcers of the skin.

Meshed and other medical graft constructs of the invention may include for example a single ECM layer or may include a plurality (two or more) of ECM layers. Preferred single- or multiple-layer ECM constructs of the invention will have an overall thickness of at least about 50 microns, typically ranging from about 80 to about 1000 microns, and in certain embodiments ranging from about 100 to about 1000 microns. Relatively thick constructs, such as multiple layered ECM constructs, can provide particularly advantageous and lasting collagen scaffolds for tissue ingrowth, especially in the field of wound care such as burn and ulcer care.

Meshed constructs of the invention will have a plurality of slits therein to provide a mesh pattern, and the mesh pattern will provide deformability to the structure, especially expandability. In this regard, in the preferred meshed constructs, expansion or other deformation of the meshed structure will widen the openings created by the slits of the mesh pattern, by lateral and/or vertical displacement of the edges of the slits relative to one another. Preferred devices of the invention will have a mesh pattern providing an expansion ratio of at least about 1.2:1 when the layer is completely hydrated, more preferably at least about 2:1, and most preferably at least about 3:1.

Medical graft devices of the invention can be used in grafting applications for treatment of human or other animal conditions. In one preferred application, the materials of the invention are used in the treatment of wounds and in particular open, cutaneous wounds. Open, cutaneous wounds may be classified into one of four grades depending on the depth of the wound. A Grade I wound is limited to the epithelium. A Grade II wound extends into the dermis. A Grade III wound extends into the subcutaneous tissue; and, a Grade IV wound (or full-thickness wound) exposes bone. The term “partial thickness wound” refers to wounds that encompass Grades I-III; examples of partial thickness wounds include burn wounds, pressure sores, venous stasis ulcers, and diabetic ulcers. Advantageous applications of products of the invention include the treatment of partial thickness open cutaneous wounds, including burns and ulcers. These wounds are often chronic (e.g. lasting at least about 30 days untreated), and benefit significantly from the application of graft products of the present invention.

In use for wound care, the physician, veterinarian or other user of the medical graft materials of the invention will prepare the wound for treatment in a conventional fashion, which may for example include cleaning and/or debridement of the wound with water, physiologic saline or other solutions, and potentially also treating the wound with antibiotics or other therapeutic agents. The medical graft construct of the invention will be applied to the wound in a fashion to facilitate and promote healing of the wound. In this regard, the inventive construct may be applied in a dehydrated, partially hydrated, or fully hydrated state. Once applied to a wound, the modified ECM graft material of the invention will hydrate (if not previously hydrated) and remain generally in place either alone or in combination with other wound dressing materials applied below or on top of the modified ECM material

The invention also encompasses medical products that include a modified ECM graft material as described herein sealed within sterile medical packaging. The final, packaged product is provided in a sterile condition. This may be achieved, for example, by gamma, e-beam or other irradiation techniques, ethylene oxide gas, or any other suitable sterilization technique, and the materials and other properties of the medical packaging will be selected accordingly. In addition, the modified ECM graft materials may be packaged in a wet or dried state. In situations wherein sensitive growth factors or other bioactive proteins native to the ECM material or added as exogenous materials are present, terminal sterilization methods that result in the retention of substantial amounts of the original activity of these materials will be preferred. In these regards, in certain inventive embodiments, packaged, modified ECM materials of the invention will be terminally sterilized using radiation such as E-beam, gas plasma (e.g. Sterrad), or hydrogen peroxide vapor processing.

For the purpose of promoting a further understanding of the present invention, the following specific Experimental is provided. It will be understood that this Experimental is illustrative, and not limiting, of the invention.

### EXPERIMENTAL

Materials and Methods

Multiple lots of OASIS® Wound Matrix (a freeze-dried sheet of small intestinal submucosa (SIS) with fenestrations available from Cook Biotech Inc., West Lafayette, Ind.) were stored in sealed sterile packaging at room temperature for an average of four months prior to experimentation. Approximately one-inch by one-inch samples of OASIS® Wound Matrix were cut from six production lots and then weighed to determine the dry weight to the nearest 0.1 mg. Duplicate samples from each of at least four lots were used for analysis. All samples were placed without rehydration into incubation solutions.

Heparin, bovine serum albumin (BSA), plasmin, and 3,3′,5,5′ tetramethylbenzidine (TMB) were purchased from Sigma Chemical Co. (St. Louis, Mo.). Biotinylated bovine albumin and horseradish peroxidase (HRP)-linked streptavidin, of the Immunopure line, were purchased from Pierce Endogen (Rockford, Ill.). Recombinant human fibronectin was acquired from Fibrogenex (Chicago, Ill.). The enzyme linked immunosorbant assay for fibronectin was purchase from Chemicon International (Temecula, Calif.). Horse serum was purchased from American Type Culture Collection (ATCC) (Manassas, Va.).

Absorption and Elution of Bioactive Molecules

Samples were incubated separately in 2 ml solutions of individual bioactive molecules at room temperature overnight with gentle agitation. Solutions contained one of the following concentrations of bioactive molecules in high purity water: 200 μg/ml BSA containing 0.1% biotinylated BSA, 20 μg/ml heparin, or 10 μg/ml fibronectin. After incubation, each sample was washed three times (4 ml each) with phosphate buffered saline solution (PBS) for one hour at room temperature with shaking to elute any unbound bioactive molecules. Each washing solution as well as each original incubation solution was collected and assayed for the respective bioactive molecule content. The residual amount of bioactive molecules in each washed sample, with the exception of the heparin samples, was also determined after homogenization in PBS at room temperature with dilution as necessary. Fibronectin levels were determined by ELISA. Heparin was assayed using a dye-mediated spectrophotometric assay and did not require homogenization, and BSA content was determined through detection of biotin content.

Spectrophotometric determination of concentrations was performed by microplate reader, linked by KC Junior interface software (Bio-tek Instruments, Inc., Winooski, Vt.). Samples for each molecule included initial, doped, and doped and rinsed SIS, as well as doping solution, post-doping solution, and three rinses. Concentrations for albumin, heparin, and fibronectin were expressed in μg/g of OASIS® Wound Matrix.

Bioactive Molecule Detection

Albumin:

BSA was quantitated by detection of biotinylated albumin, spiked at 0.1% of total BSA, using horseradish peroxidase (HRP)-linked streptavidin. TMB (3,3′,5,5′ tetramethylbenzidine) was used as the reporter substrate for peroxidase. Collected samples (200 μl) were placed in wells of a 96-well polystyrene (high protein binding) microplate for two hours at room temperature to bind the BSA to the surface. After removal of the sample, each well was blocked at room temperature for one hour, followed by three rinses with PBS (200 μl). HRP-linked streptavidin (0.1 g/ml) was added to each well and incubated at room temperature for one hour with agitation. After incubation, each well was rinsed three times with PBS (200 μl), followed by addition of the TMB substrate (100 μl), development for 30 minutes at room temperature, and reaction stopping with sulfuric acid (50 μl). Samples were read at 450 mm and compared to a standard curve of biotinylated albumin concentrations ranging from 1 ng/ml to 1 μg/ml.

Heparin:

Heparin in the samples was determined using a dye-mediated detection assay.^{20,21} Briefly, samples were incubated in toluidine blue (2 mg/5 ml) overnight at room temperature with gentle agitation. The dicationic dye forms a complex with heparin, which appears purple. Samples were then washed thoroughly with high purity water to remove excess dye. The resulting bound toluidine blue was solubilized by incubating the samples in a mixture (1:4) of NaOH (0.1N) and ethanol. The sample content of solubilized dye was read at 530 nm and compared to a standard curve of heparin at concentrations ranging from 3 μg/ml to 13 μg/ml.

Heparin in solution was measured using another dye-mediated detection assay, which involved the color shift of a heparin-Azure A complex.^{22,23} One milliliter of an Azure A solution (0.07 mg/ml) was added to one milliliter of solution samples, followed by brief vortexing and plating into a 96-well microplate. Sample absorbances were read at 620 nm and compared to a standard curve of heparin with concentrations ranging from 4 μg/ml to 14 μg/ml.

Fibronectin:

Fibronectin levels were determined using a Quantimatrix ELISA kit. The ELISA was performed according to the manufacturer's protocol. Sample absorbances were read at 450 nm and compared to a standard curve of fibronectin with concentrations ranging from 3 ng/ml to 1000 ng/ml.

Statistical Analysis

All results are given as mean±standard error of the mean. Statistical differences were determined using pair-wise t-tests. A p-value of <0.05 was considered significant.

Results

Heparin, albumin, a ubiquitous protein in serum, and fibronectin, a glycoprotein, are commonly found in wound fluid and are representative molecules of the wound environment. When approximately one-inch square samples of OASIS® were incubated overnight in solutions of heparin, albumin or fibronectin solutions, significant increases of these bioactive molecule contents were observed, as shown in Table 1 below.

TABLE 1
OASIS ® Wound Matrix Absorbs Albumin, Heparin, and Fibronectin.

| Bioactive | Initial Amount | Post-Incubation | Fold Increase | Post-Elution | Fold Increase | Retention |
|---|---|---|---|---|---|---|
| Molecule | (μg/g) | Amount (μg/g) | over Initial | Amount (μg/g) | over Initial | Percentage |
| Heparin | 437 ± 39 | 986 ± 110* | 2.3 | 446 ± 41 | 1.02 | 0.9% |
| Albumin | 363 ± 61 | 6428 ± 1429* | 17.7 | 3608 ± 662* | 9.9 | 50.5% |
| Fibronectin | 1.09 ± 0.07 | 27.0 ± 2.1* | 24.8 | 3.83 ± 0.51* | 3.5 | 10.1% |

*p < 0.05 vs. initial

Comparison of the initial OASIS® Wound Matrix vs. post-incubation OASIS® Wound Matrix indicated a 2-fold increase in heparin content (437 μg/g±39 μg/g initial vs. 986 μg/g±111 μg/g post-incubation, p<0.001), a nearly 18-fold increase in albumin content (363 μg/g±61 μg/g initial vs. 6,428 μg/g±1,429 μg/g post-incubation, p<0.01), and a nearly 25-fold increase in fibronectin content (1.09 μg/g±0.07 μg/g initial vs. 27.0 μg/g±2.1 μg/g post-incubation, p<0.005). After three rinses in PBS, heparin content decreased back to initial levels. The albumin and fibronectin bound to OASIS® Wound Matrix samples decreased to 10-fold and 3.5-fold, respectively, above (initial) untreated OASIS® Wound Matrix. While the majority of heparin and fibronectin absorbed was removed through elution rinses, OASIS® Wound Matrix samples still retained 50% and 10% of the absorbed albumin and fibronectin, respectively (Table 1).

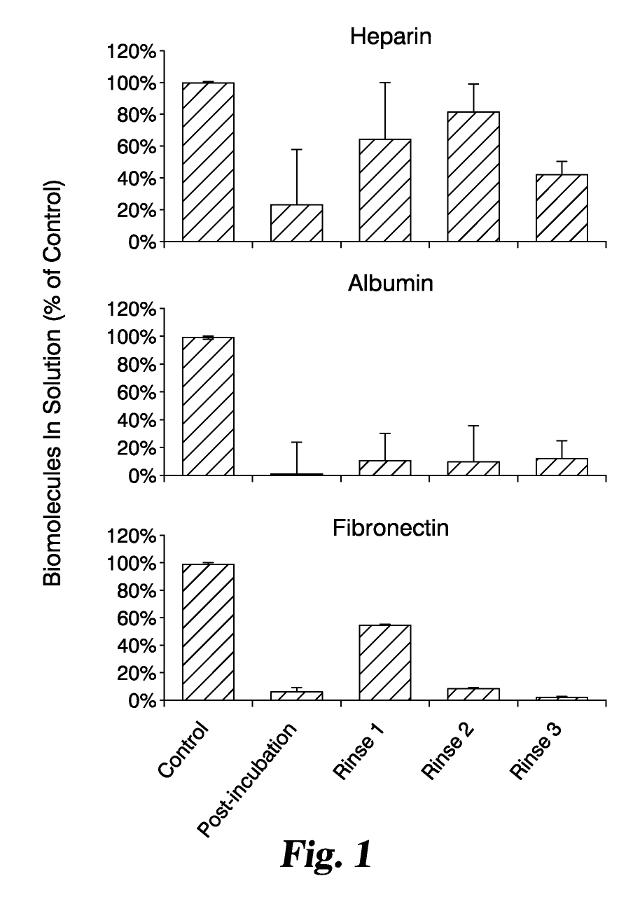

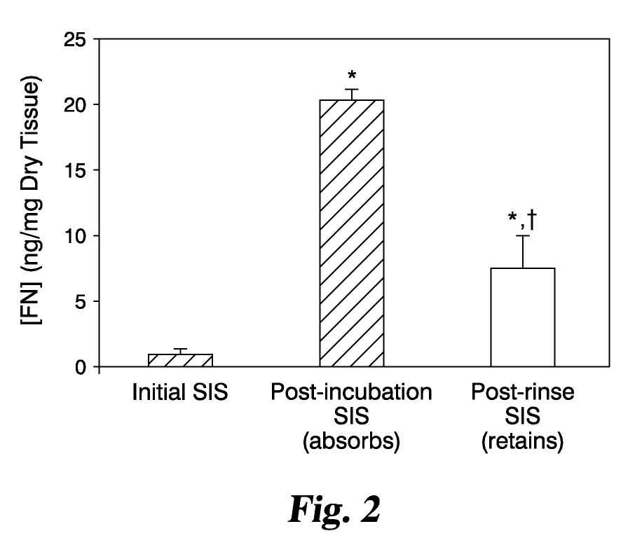

Measurements of heparin, albumin, and fibronectin in the initial bioactive molecule solutions after incubation indicated that a significant amount was absorbed by the OASIS® Wound Matrix samples. The rinse solutions for these three biomolecules were evaluated. Heparin was significantly released into each of the rinse solutions, suggesting loose non-specific absorption into OASIS® Wound Matrix (FIG. 1). Consistently low albumin concentrations were found in each of the rinse solutions, suggesting that OASIS® Wound Matrix bound albumin in a non-specific manner. Fibronectin displayed decreasing release with subsequent rinses indicating initial loss of loosely absorbed fibronectin without removal of strongly bound fibronectin, likely due to specific binding. Thus, upon analysis, OASIS® Wound Matrix samples were able to retain substantial bound fibronectin even after repeated rinsing (FIG. 2). Such materials can be processed to sterile conditions and used as medical graft materials, or can be used effectively as precursor materials to be modified with one or more additional exogenous bioactive substances as taught in the descriptions hereinabove.

While the invention has been illustrated and described in detail in the foregoing description, the same is to be considered as illustrative and not restrictive in character, it being understood that only certain embodiments have been shown and described and that all changes and modifications that come within the spirit of the invention are desired to be protected. In addition, all publications cited herein are hereby incorporated by reference in their entirety as if each had been individually incorporated by reference and fully set forth.

What is claimed is:

1. A medical graft material, comprising:
submucosa; and
an extracellular matrix material (ECM) comprising exogenous fibronectin; and
wherein said exogenous fibronectin is bound to said ECM; and
wherein said submucosa is lyophilized.

2. The material of claim 1 wherein at least 50% of the exogenous fibronectin incorporated in or on the ECM is bound to the ECM.

3. The material of claim 1 wherein at least 75% of the exogenous fibronectin incorporated in or on the ECM is bound to the ECM.

4. The material of claim 1 wherein at least 90% of the exogenous fibronectin incorporated in or on the ECM is bound to the ECM.

5. The material of claim 1 wherein essentially all of the exogenous fibronectin incorporated in or on the ECM is bound to the ECM.

6. The material of claim 1 and also including another exogenous bioactive material bound to the exogenous fibronectin.

7. The material of claim 6 wherein the other exogenous bioactive material bound to the exogenous fibronectin comprises heparin and/or heparin sulfate.

8. The material of claim 7 and also including another bioactive material bound to the heparin and/or heparin sulfate.

9. The material of claim 8 wherein the other bioactive material bound to the heparin and/or heparin sulfate is a growth factor.

10. A method for preparing a medical graft material, comprising:
first contacting an extracellular matrix material (ECM) comprising submucosa with a liquid medium containing fibronectin so as to prepare a modified ECM material incorporating fibronectin that is bound to the ECM and fibronectin that is not bound to the ECM; and
rinsing the modified ECM to remove at least a portion of the fibronectin that is not bound to the ECM.

11. The method of claim 10 also including second contacting the submucosa after said rinsing with heparin and/or heparin sulfate.

12. The method of claim 11 also including third contacting the ECM with a heparin-binding growth factor after said second contacting step.

13. A medical graft material, comprising:
a remodelable collagenous extracellular matrix material (ECM); and
exogenous fibronectin molecules incorporated in or on the ECM, wherein at least 50% of the exogenous fibronectin incorporated in or on the ECM is bound to the ECM.

14. The material of claim 13 wherein the ECM is lyophilized.

15. The material of claim 13 wherein at least 75% of the exogenous fibronectin incorporated in or on the ECM is bound to the ECM.

16. The material of claim 13 wherein at least 90% of the exogenous fibronectin incorporated in or on the ECM is bound to the ECM.

17. The material of claim 13 wherein essentially all of the exogenous fibronectin incorporated in or on the ECM is bound to the ECM.

18. The material of claim 13 and also including another exogenous bioactive material bound to the exogenous fibronectin.

19. The material of claim 18 wherein the other exogenous bioactive material bound to the exogenous fibronectin comprises heparin and/or heparin sulfate.

20. The material of claim 19 and also including another bioactive material bound to the heparin and/or heparin sulfate.

21. The material of claim 20 wherein the other bioactive material bound to the heparin and/or heparin sulfate is a growth factor.

22. The material of claim 13 wherein the ECM comprises submucosa.

23. A method for preparing a modified extracellular matrix material, comprising:
providing an extracellular matrix material;
contacting the extracellular matrix material with an amount of exogenous fibronectin so as to prepare a first modified extracellular matrix material having fibronectin molecules bound to the extracellular matrix material;
rinsing the modified extracellular matrix material to remove at least a portion of the fibronectin that is not bound to the material; and
contacting the first modified extracellular matrix material with an amount of exogenous heparin and/or heparin sulfate so as to prepare a second modified extracellular matrix material having exogenous heparin and/or heparin sulfate molecules bound to the exogenous fibronectin bound to the extracellular matrix material.

24. The method of claim 23, also comprising contacting the second modified extracellular matrix material with an amount of a bioactive substance that binds to the heparin and/or heparin sulfate, so as to prepare a third modified extracellular matrix material having molecules of the bioactive substance bound to the exogenous heparin.

25. The method of claim 23, wherein the bioactive substance comprises a growth factor.

26. A modified collagenous matrix material, comprising:
a collagenous matrix scaffold;
fibronectin molecules incorporated in or on the collagenous matrix scaffold wherein at least 50% of the exogenous fibronectin incorporated in or on the scaffold is bound to the scaffold; and
heparin and/or heparin sulfate molecules bound to said fibronectin molecules.

27. The matrix material of claim 26, also comprising a heparin-binding protein bound to said heparin or heparin sulfate molecules.

28. The matrix material of claim 27, wherein said collagenous matrix scaffold comprises a collagenous sheet material isolated from animal tissue.

29. The matrix material of claim 27, wherein said collagenous scaffold comprises reconstituted or electroprocessed collagen fibers.

30. The matrix material of claim 28, wherein said collagenous sheet material consists essentially of collagen or of collagen and elastin.

31. The matrix material of claim 28, where said collagen sheet material retains at least one bioactive component native to the animal tissue.

32. The matrix material of claim 26, wherein the collagenous matrix material is lyophilized.

33. The material of claim 26, also comprising a growth factor that promotes wound healing bound to the heparin or heparin sulfate.
